(19) United States
(12) Patent Application Publication
(10) Pub. No.: US 2026/0283815 A1
(43) Pub. Date: Sep. 24, 2026

(54) Intervertebral Disc Replacement Implant And Methods Of Use

(71) Applicant: Brian R. Perri, Los Angeles, CA (US)

(72) Inventor: Brian R. Perri, Los Angeles, CA (US)

(21) Appl. No.: 19/573,873

(22) Filed: Mar. 20, 2026

Related U.S. Application Data
(60) Provisional application No. 63/775,108, filed on Mar. 20, 2025.

(51) Int. Cl. A61F 2/44; A61F 2/30

(52) U.S. Cl. CPC A61F 2/4425; A61F 2/30771; A61F 2002/30113; A61F 2002/30245; A61F 2002/30273; A61F 2002/30324; A61F 2002/30326; A61F 2002/30649; A61F 2002/30843; A61F 2002/30884; A61F 2002/30892; A61F 2002/30894; A61F 2002/3093; A61F 2002/443

(57) ABSTRACT

Artificial disc replacement implants for use in C-ADR, AL-ADR, or LL-ADR comprising a cephalad endplate, a caudal endplate, and a core disposed therebetween are disclosed along with methods for replacing one or more damaged discs. The cephalad endplate has a domed portion, while the core's superior portion has a convex surface. Both endplates have textured or porous regions and fixation elements to facilitate fixation and bone ongrowth or ingrowth. The implant is designed for cervical or lumbar spine insertion. Fixation elements include keels or spikes configured to facilitate insertion of the implant without a chiseling step prior to insertion.

### CROSS REFERENCE TO RELATED APPLICATION

This application claims priority to U.S. provisional application No. 63/775,108, filed Mar. 20, 2025, the disclosure of which is incorporated herein by reference.

### FIELD OF THE INVENTION

The disclosed invention is generally in the field of intervertebral disc replacement.

### BACKGROUND OF THE INVENTION

Currently available FDA approved arthroplasty endplate devices are first manufactured then receive a secondary step to apply a plasma spray titanium coating on the surface effacing the patient's bone. The process of “bonding” the plasma spray surface coating presents a risk for delamination of titanium particles which has been shown to result in an inflammatory host response causing local osteolysis (Torstrick et al.).

Typical failure of disc implants is commonly a progression resulting from component wear debris, subsidence, dislocation or device migration. There are many additional causes for artificial disc implant failure, not only component breakdown, but also mispositioning of implant, peri-prosthetic vertebral fracture and poor patient selection. Typical causes of failure come from the device components becoming loosened and the failure to support the load required, such as due to lordosis, i.e. the difference between the anterior and posterior height.

Restoration of sagittal balance is a critical element in the treatment of spinal deformities. Yet most current FDA-approved implants do little to restore sagittal alignment. The normal curvature of the cervical and lumbar spine is in lordosis, globally and for each spinal segment.

Again, the wear of all articulating device components is of critical importance. This not only includes the backside surface wear of the plasma spray titanium coating (Torstrick et al.) but also wear of the articulating endplates and central core (Veruva et al.). Endplate wear debris has been identified in retrieved implants from revision surgeries (Kurtz et al. and Darren et al.). This material debris from wear has been shown to cause implant function failure by triggering a local host inflammatory response. This may lead to alterations in implant motion, dislocation from the insertion site (which may result in neurologic injury) or subsidence. When third-body wear, due to the presence of foreign particles, such as bone cement debris, trapped between the implant surfaces during movement results in osteolysis of surrounding host bone, there can be subsidence of the implant or loss of normal lordotic sagittal alignment of the cervical spine (Scott-Young et al.).

Therefore, there is a need for cervical artificial discs (C-ADR) and anterior lumbar artificial discs (AL-ADR) that more closely mimic a normal disc height, angulation and/or that more closely mimic change of the normal motion pattern of the instant center of rotation (ICR) from flexion to extension and side bending for both cervical artificial discs (C-ADR) and anterior lumbar artificial discs (AL-ADR).

Additionally, there is a need for improved C-ADRs and AL-ADRs ability to correct sagittal alignment.

Additionally, there is a need for cervical artificial discs (C-ADR) and anterior lumbar artificial discs (AL-ADR) with improved initial and long-term fixation within the spine.

There is a further need for improved surgical methods for inserting cervical artificial discs (C-ADR) and anterior lumbar artificial discs (AL-ADR).

It is an object of the invention to provide improved artificial discs for C-ADR, AL-ADR, and/or left lateral artificial disc replacement (LL-ADR) that meet one or more of these needs.

It is a further object of the invention to provide improved methods for replacing a damaged cervical or lumbar disc in a patient.

### BRIEF SUMMARY OF THE INVENTION

Artificial disc replacement implants comprising a cephalad endplate, a caudal endplate, and a core are disclosed herein. The core may be disposed between the cephalad endplate and the caudal endplate. The cephalad endplate may comprise a domed portion. The superior portion of the core may have a convex surface configured to fit inside the domed portion of the cephalad endplate. The superior surface of the cephalad endplate and the inferior surface of the caudal endplate may comprise one or more textured or porous regions to facilitate bone ongrowth and/or ingrowth and one or more fixation elements. The caudal endplate includes an anterior end and a posterior end, optionally the anterior end has a height that is greater than the height of the posterior end. The caudal endplate may have a lordotic angulation in the range of about 0 degrees to about 9 degrees.

The one or more fixation elements attached to the superior surface of the cephalad endplate and/or the inferior surface of the caudal endplate may be keels, spikes, or a combination thereof. The one or more fixation elements may comprise a sharp leading edge at the posterior aspect of the fixation element, and may widen toward the anterior end of the implant. Optionally, the fixation elements include two or more keels or two or more spikes.

The concavity of the inferior surface of the concave portion of the cephalad endplate may match the convexity of the superior portion of the core, such that the core may fit concentrically inside the concavity in a ball-and-socket configuration. Alternatively, the concavity of the inferior surface of the cephalad endplate may be offset, in anonconcentric design, with an elongation slightly anteriorly and posteriorly from the outer anterior and posterior margins of the core, respectively; and in a similar manner, the nonconcentric design may include a slight widening on both sides of the concavity in the mid coronal plane. More specifically, when the implant is viewed in neutral alignment, these gaps creating the offsets in the cervical disc replacement can be in the range of 0.67 mm anterior and 0.33 mm posterior at the midsagittal plane and 0.25 mm on both sides viewed in the mid coronal plane. With the lumbar disc replacement implant, these measurements will be in the range of 1.3 mm anterior elongation of the concavity, 0.67 mm posterior elongation of the concavity in the midsagittal plane and 0.5 mm widening of the concavity on each side at the mid coronal plane.

A method for replacing a damaged cervical or lumbar disc in a patient may comprise, after the damaged disc is removed from between adjacent vertebrae, inserting the implant into the intervertebral space. Optionally, the method does not include a chiseling or drilling step to prepare the surfaces of the vertebral bodies prior to inserting the implant into the disc space.

Additional advantages of the disclosed implant, system, and kit will be set forth in part in the description which follows, and in part will be understood from the description, or can be learned by practice of the disclosed method and compositions. The advantages of the disclosed implant, system, and instrumentation will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings illustrate exemplary embodiments of the disclosed implant and components thereof.

FIGS. 1A-E are schematics of different views of an exemplary cervical artificial disc implant, containing keels as the fixation elements. FIG. 1A is a perspective view of the top.

FIG. 1B is a side view of the anterior face. FIG. 1C shows a cross-sectional view taken about line B-B of FIG. 1B. FIG. 1D shows a side view. FIG. 1E shows a bottom view of the caudal endplate of the implant.

FIGS. 2A and 2B are schematics of different views of an exemplary cervical artificial disc implant with spikes as the fixation elements. FIG. 2A is a top perspective view. FIG. 2B shows a posterior face view.

FIGS. 3A and 3B are schematics showing the same cross-sectional view, mid-sagittal view, for two different exemplary cervical artificial disc implants when the components of the implant are in a neutral alignment. FIG. 3A is a schematic of an exemplary cervical artificial disc implant where the concavity of the domed portion of the cephalad endplate is elongated compared to the curvature of the superior portion of the core, such that there is a gap having a length (g) between the surface of the core and the corresponding inner surface of the domed portion at the anterior end of the implant. Additionally, there is a gap on each side of the concavity of the domed portion relative to the superior portion of the core when viewed in the mid coronal plane (not shown in FIG. 3A). FIG. 3B is a schematic of an exemplary cervical artificial disc implant where the concavity of the interior surface of the domed portion of the cephalad endplate concentrically matches the convexity of the core, such that the core fits inside the domed portion in a ball-and-socket configuration (i.e. no gap is present between the inner surface of the domed portion and the superior portion of the core.

FIGS. 4A and 4B are two views of an exemplary cervical artificial disc implant with keels as the fixation elements and illustrating the porous surface coating for promoting bone ingrowth and ongrowth. FIG. 4A is a top perspective view with the posterior end in the foreground. FIG. 4B is a bottom perspective view with the posterior end in the foreground.

FIGS. 5A and 5B are illustrations showing the shape and positions of different types of keels in a C-ADR relative to the spinal cord in a patient. FIG. 5A shows an existing C-ADR, the prodisc C, having keels protruding from the cephalad endplate andsother keels protruding from the caudal endplate, where each keel has a substantially rectangular profile when observed laterally in a plane adjacent to the sagittal plane. FIG. 5B shows an exemplary C-ADR as described herein, which contains keels protruding from the cephalad endplate and the caudal endplate, where each keel has a profile that curves away from the spinal cord when observed laterally in a plane adjacent to the sagittal plane.

FIGS. 6A and 6B are two views of an exemplary cervical artificial disc implant with keels as the fixation elements, as depicted in FIGS. 4A and 4B, however the interior surfaces of each endplate is coated with TiN (titanium nitride) to reduce friction and improve wear resistance between the core and the corresponding articulating surface on the cephalad or caudal endplate. FIG. 6A shows the endplates separate from each other, to show the interior concave surface of the cephalad endplate, which articulates with the core when implanted in a subject. FIG. 6B shows the implant with the endplates in a closed position.

FIGS. 7A and 7B are schematics showing two cross-sectional views for the same exemplary cervical artificial disc implants with a nonconcentric design, that results in offsets in the concavity of the domed portion relative to the convexity of the superior surface of the core when the components of the implant are in a neutral alignment. FIG. 7A shows midsagittal view of the cross-section, and FIG. 7B shows an anterior view of a cross section taken at the mid-coronal plane about line AF of FIG. 7A. FIGS. 7A and 7B each include two magnified sections to illustrate the different size gaps of the offsets that can be present in an implant with a nonconcentric design.

FIGS. 8A and 8B are schematics showing the same cross-sectional view of the mid-sagittal plane, for two different exemplary cervical artificial disc implants when the components of the implant are in a neutral alignment. FIG. 8A is a schematic of an exemplary cervical artificial disc implant where the concavity of the domed portion of the cephalad endplate is nonconcentric and elongated relative to the outer anterior and posterior margins of the core. Thus, the anterior elongation relative to the superior surface of the core can be measured where the offset is the greatest as a gap having a length (g₁) between the surface of the core and the adjacent inner surface of the domed portion at the anterior end of the implant. Additionally, there is a gap measured where the offset is the greatest having a length (g₂) between the surface of the core and the corresponding inner surface of the domed portion at the posterior end of the implant. FIG. 8B is a schematic of an exemplary cervical artificial disc implant where the concavity of the interior surface of the domed portion of the cephalad endplate concentrically matches the convexity of the core, such that the core fits inside the domed portion in a ball-and-socket configuration (i.e. no gap is present between the inner surface of the domed portion and the superior portion of the core. As shown on FIGS. 8A and 8B, the core can be recessed to a depth d₂ and d₃, respectively, into the interior surface of the domed portion of the cephalad endplate.

### DETAILED DESCRIPTION OF THE INVENTION

The disclosed implants, systems and methods can be understood more readily by reference to the following detailed description of particular embodiments and the Example included therein and to the Figures and their previous and following description.

It is to be understood that the disclosed implants, systems and methods are not limited to specific materials and structures described herein unless otherwise specified, and, as such, can vary. It is also to be understood that the terminology used herein is for the purpose of describing particular embodiments only and is not intended to be limiting.

Throughout this specification the word “comprise,” or variations such as “comprises” or “comprising,” will be understood to imply the inclusion of a stated element or step, or group of elements or steps, but not the exclusion of any other element or step, or group of elements or steps.

### A. Implants

The implant generally includes a first endplate, a second endplate, a core disposed between the first endplate and the second endplate and moveably coupled thereto. The first and second endplates include one or more fixation elements configured to contact and become partially embedded in the superior vertebral body or the inferior vertebral body, respectively. The first endplate can be a cephalad endplate and the second endplate can be a caudal endplate. The same or different types, sizes and/or shapes of fixation elements can be included on the bone contacting surface of each endplate.

In addition to the features shown in the Figures, the endplates may contain slots and/or channels to mate with instruments to facilitate holding and inserting the implants. In addition, the insertion instrument may be registered such that robotic navigation can be employed to improve accuracy of midline placement and a tangential insertion of the implants disclosed herein, such as cervical, anterior lumbar and/or lateral lumbar implants for replacing a damaged cervical or lumbar disc in a patient.

Optionally, the core contains a radiopaque marker or material to facilitate visualization during imaging.

### 1. Core

The implant described herein includes a core disposed between the first endplate, i.e. the cephalad endplate, and the second endplate, i.e. the caudal endplate. The core includes an inferior end which is affixed to the interior surface of the caudal endplate. The core is made from any suitable, biocompatible material. In some embodiments, the core includes ultra-high molecular weight polyethylene (UHMWPE), ceramic polymer, and/or any other suitable material that has a suitable wear resistance according to standard biomechanical testing. The core can be manufactured using standards and methods known in the field with any suitable material for manufacturing, sterilizing, and/or conditioning. In some instances, the core is sterilized and conditioned with vitamin E to improve component integrity and longevity. In some instances, the method of manufacturing improves material's oxidation resistance, wear resistance, and fatigue strength (Ebru Oral & Orhun K. Muratoglu).

In some embodiments, the core is manufactured separately from the endplates, then inserted into the caudal endplate and affixed thereto. Prior to insertion in the patient's spine, the cephalad endplate is placed on top of the core. Following insertion and initial fixation of the implant in the patient's spine, the core functions by articulating with the undersurface of the cephalad endplate.

The core includes a convex upper portion configured to be received by the domed portion of the cephalad endplate and a generally flat inferior portion configured to be received by the interior surface of the caudal endplate. Optionally the inferior portion of the core is configured to slide in one or more slots in the interior surface of the caudal endplate. Optionally the inferior portion of the core is configured to be received in a depression in the interior surface of the caudal endplate in a friction fit relation thereto.

### 2. Endplates

a. Cephalad Endplate

The cephalad endplate includes a superior surface and an interior, undersurface on the opposite side of the endplate. The superior surface of the cephalad endplate include a central dome forming a convex surface (“domed portion”) that generally matches the surface of the superior vertebral body's inferior endplate. The domed portion of the cephalad endplate that effaces the host bone generally contours with the anatomic detail of the undersurface of the vertebrae. However, if the patient has a generally flat inferior vertebral endplate, a milling device may be used to create a concavity that is configured to accommodate the domed portion of the cephalad ADR endplate.

The central domed portion is optionally surrounded by a substantially flat flange. The superior surface of the cephalad endplate optionally has a porous surface for the vertebral bones to attach to and integrate with, thereby promoting bone ongrowth and/or ingrowth. Optionally, one or more fixation elements protrude superiorly from the superior surface of the flange. Optionally one or more fixation elements protrude from the domed portion.

The domed portion of the cephalad endplate which effaces the host bone contours with this common anatomic detail of the undersurface of the inferior vertebral endplate.

The undersurface of the cephalad endplate faces and contacts the artificial disc core and is generally smooth and concave.

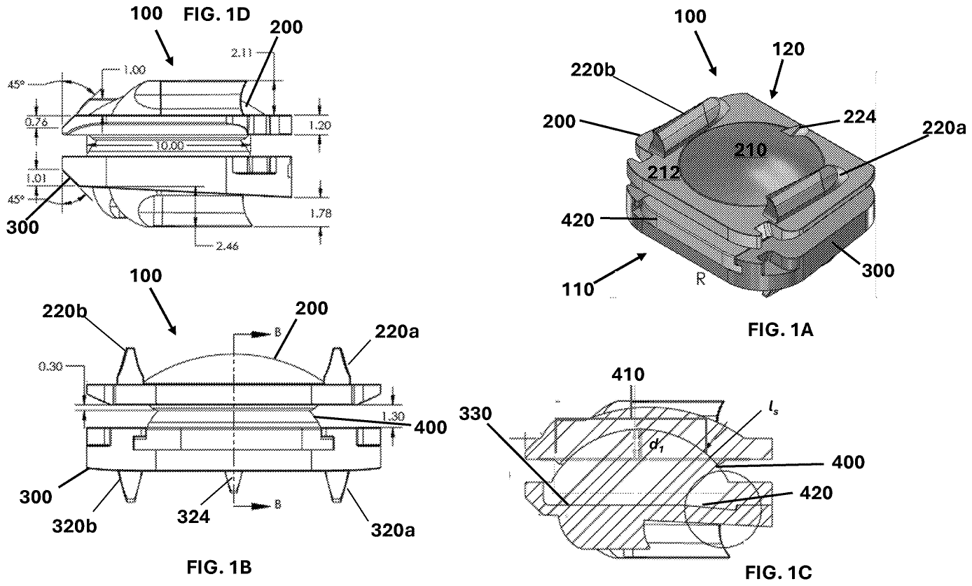

The domed portion of the cephalad endplate has a suitable size and shape to receive the core such that the superior portion of the core fits inside the dome at a depth (d), measured when viewing a cross-section of the implant (see, e.g. FIG. 1C). Optionally the depth d ranges from about 1-2 mm. This arrangement allows for lower overall prosthesis heights compared to currently available C-ADRs or AL-ADRs.

In some embodiments, the concavity of the undersurface of the cephalad endplate matches the convexity of the core, such that the core fits inside the domed portion concentrically, i.e. in a ball-and-socket configuration. In these embodiments, due to the close fit of the core inside the domed portion of the cephalad endplate, the core remains stationary and the cephalad endplate articulates with minimal translation around the core during flexion, extension, rotation, and/or side bending by the patient.

In other embodiments, the interior diameter of the domed portion is greater than the diameter of the core, such as at least 2-5% greater, up to about 10% greater. Having a domed portion that has a sufficiently wider diameter than the diameter of the core, yet a similar curvature allows the cephalad endplate, and thus the patient's cephalad vertebrae, to translate posteriorly with cervical and lumbar extension of the patient's spine for the C-ADR and AL-ADR embodiments, respectively.

b. Caudal Endplate

The caudal endplate has a superior surface that contacts the core and is fixed thereto, and an inferior surface that contacts and fixes to the inferior vertebral body, following insertion into the spine. Optionally the superior surface includes a central depression that is configured to receive the bottom surface of the core. Optionally the central depression has substantially the same size and shape as the bottom surface of the core, such that the bottom surface of the core fits inside the central depression of the interior surface of the caudal endplate.

The caudal endplate can have different angulations to accommodate the differences in disc space angulation in each patient. In some instances, the anterior side of the caudal endplate is taller than the posterior side of the caudal endplate, which provides an angulation of greater than 0 degrees, such as an angulation of 3 degrees, or 6 degrees.

The inferior surface of the caudal endplate optionally has a porous and/or textured surface configured to promote bone ongrowth and/or ingrowth. Optionally one or more fixation elements protrude inferiorly from the exterior surface of the caudal endplate.

c. Materials

The endplates of the implant can be manufactured using a suitable medical-grade, inert material with sufficient strength and durability while allowing for improved visualization of the surrounding anatomy when evaluated postoperatively by X-ray, CT scan, and/or MRI compared to the same endplate when formed of a cobalt-chromium molybdenum alloy. Suitable materials for forming the endplates include titanium-ceramic polymers, such as Ti-6Al-4V, e.g. grade 23; commercially pure titanium (CPT); inert, biocompatible polymers, such as polyether-ether-ketone (PEEK), carbon-fiber polyether-ether-ketone (CF-PEEK), or an inert, biocompatible ceramic alloy.

The endplate surface for both the caudal and cephalad endplates effacing the patient's bone has a porous surface configured to promote bone ongrowth and/or ingrowth and is formed of or includes a titanium containing material, such as commercially pure titanium (CPT) or a titanium-ceramic polymer, such as Ti-6Al-4V The material that is used to form this surface coating of each of endplate is typically the same as the material used to form the rest of the endplate.

The endplates, including their surface coating, can be manufactured via additive manufacturing using 3-dimensional printing, such as described in Fatma et al., J. of Mat. Res. and Tech., 2022; 17; 1408-1426. Thus, in some instances, the endplate is formed using a one-step process via three-dimensional, additive manufacturing.

In other instances, the endplate is formed of a non-metal material, such as polyether-ether-ketone (PEEK) or carbon-fiber polyether-ether-ketone (CF-PEEK), and as a separate step, a plasma titanium material is sprayed onto the surface of the endplate that contacts a patient's bone and is bonded thereto. The plasma spray coating allows for bone ongrowth, optionally the coating promotes both bone ongrowth and ingrowth.

In other embodiments, endplates formed of CPT, Ti-6Al-4V, PEEK, CF-PEEK are coated with titanium nitride (TiN), a hard ceramic coating, using physical vapor deposition or another application method. This titanium nitride coating lowers the coefficient of friction between the articulating surface of the cephalad endplate and the superior part of the core, thereby reducing friction and improving wear resistance between the concavity of the domed portion of the cephalad endplate and the superior portion of the core.

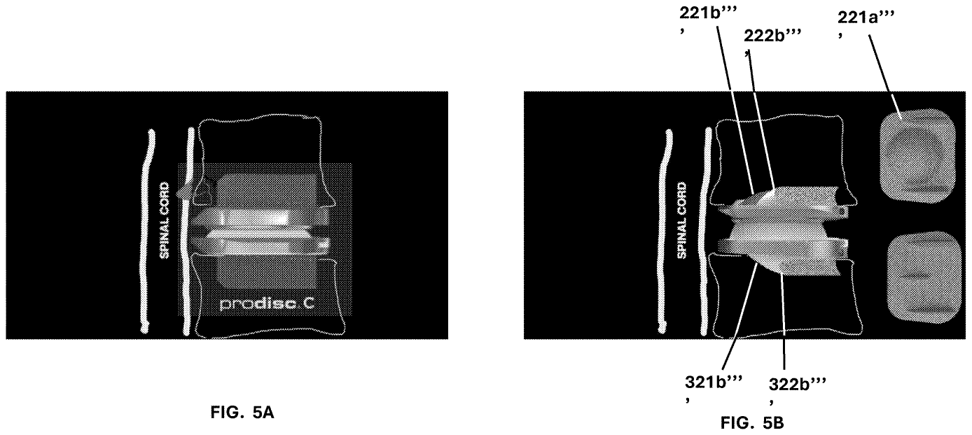

Exemplary endplates containing a titanium nitride (TiN) coating on the interior surfaces of the caudal and cephalad endplates that can contact each other or the core is illustrated in FIGS. 6A and 6B. As shown in FIGS. 6A and 6B, at least the interior surface of the domed portion 214, and optionally all of the interior surface 260 of the cephalad endplate is coated with a titanium nitride coating. Similarly, at least the flange 362 of the interior surface 360 of the caudal endplate 300 that surrounds the depression 330 is coated with a titanium nitride coating. This coating reduces friction, and thereby improves wear resistance between the core 400 and the interior surface of the domed portion 214 and between the flange 262 of the interior surface 260 of the cephalad endplate 200 and the flange 362 of the interior surface 360 of the caudal endplate 300 that surrounds the depression, when these interior surfaces contact each other.

d. Porous or Textured Bone-Contacting Surface

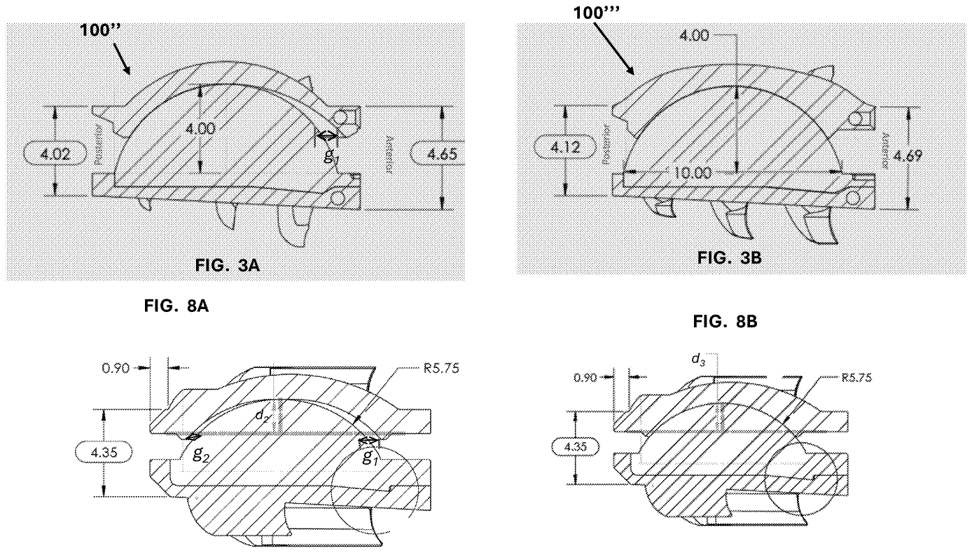

As shown in FIG. 4A, the exterior surfaces of the endplates may be textured or porous to promote attachment to and integration with the adjacent vertebral bodies. The textured or porous surfaces may facilitate bone ongrowth and ingrowth. The textured surface may comprise a series of small protrusions, indentations, or irregular features. The porous surface may contain interconnected pores or channels of varying sizes and shapes.

The textured or porous regions may cover all or portions of the exterior surfaces of the cephalad and caudal endplates that contact the vertebral bodies. These regions may be created through various manufacturing processes, such as 3D printing, or alternatively plasma spraying.

The textured or porous surfaces may be formed integrally with the endplates or may be applied as a separate coating layer. For titanium alloy endplates, the porous coating may be formed of commercially pure titanium particles sintered to the endplate surface. For PEEK endplates, a titanium plasma spray coating may be applied to create the textured surface.

These surface features may enhance initial stability of the implant by increasing friction with the vertebral endplates. Over time, the textured or porous surfaces may allow for bony ingrowth, leading to long-term fixation of the implant to the adjacent vertebrae.

e. Sizes and Shapes

The overall dimension of the endplates are selected to accommodate the patient's anatomy. For example, the endplates can have dimensions in the range of about 12-18 mm in anteroposterior diameter and about 14-18 mm in width.

i. Caudal Endplate

The caudal endplate, also referred to as the second endplate, may have a variety of different angulations to accommodate the differences in disc space angulation in each patient and/or to correct spinal alignment in patients with loss of lordosis. Suitable endplate angulations range from 0 to 9 degrees, such as 0 degrees, 3 degrees, 6 degrees, and 9 degrees of angulation. Endplates with angulation greater than 0 degrees, such as caudal endplates with 3 degree, 6 degree, or 9 degree disc space angulation have an anterior thickness that is greater than the posterior thickness. Caudal endplates with a 0 degree disc space angulation have an anterior thickness that is substantially the same as the posterior thickness.

The larger the caudal endplate's angulation, the greater the resulting improvement in lordotic sagittal alignment for the patient following implantation. The lordotic repositioning obtainable following implantation of an implanted C-ADR, LL-ADR, or AL-ADR as described herein can also provide a correction of pre-surgery disc level pathology when needed and, optionally, can be used to decrease the slope of the caudal vertebral body's superior endplate. Alternatively, following a C-ADR, LL-ADR, or AL-ADR, as described herein, the lordotic repositioning can occur by correcting kyphosis of the cephalad vertebrae relative to the caudal vertebrae.

In some instances, the implant is a C-ADR and configured to provide 3 degrees of lordosis following implantation in a patient's cervical spine. For example, the implant can include a cephalad endplate having 0 degrees of lordotic angulation and a caudal endplate having 3 degrees of lordotic angulation. In some instances, the implant is a C-ADR and is configured to provide 6 degrees of lordosis following implantation in a patient's cervical spine. For example, the implant can include a cephalad endplate having 0 degrees of lordotic angulation and a caudal endplate having 6 degrees of lordotic angulation. In some instances, no lordotic correction is needed by the patient. In these situations, neutral or zero degrees of endplate angulation will be available from the implant. For example, the implant is a C-ADR and includes a cephalad endplate having 0 degrees of lordotic angulation and a caudal endplate having 0 degrees of lordotic angulation.

In some instances, the implant is an AL-ADR or an LL-ADR and is configured to provide 3 degrees of lordosis following implantation in a patient's lumber spine. For example, the implant can include a cephalad endplate having 0 degrees of lordotic angulation and a caudal endplate having 3 degrees of lordotic angulation. In some instances, the implant is an AL-ADR or an LL-ADR and is configured to provide 6 degrees of lordosis following implantation in a patient's lumbar spine. For example, the implant can include a cephalad endplate having 0 degrees of lordotic angulation and a caudal endplate having 6 degrees of lordotic angulation. Alternatively, the implant can include a cephalad endplate having 3 degrees of lordotic angulation and a caudal endplate having 3 degrees of lordotic angulation. In some instances, the implant is an AL-ADR or an LL-ADR and is configured to provide 9 degrees of lordosis following implantation in a patient's lumbar spine. For example, the implant can include a cephalad endplate having 3 degrees of lordotic angulation and a caudal endplate having 6 degrees of lordotic angulation. In some instances, the implant is an AL-ADR or an LL-ADR and is configured to provide 12 degrees of lordosis following implantation in a patient's lumbar spine. For example, the implant can include a cephalad endplate having 3 degrees of lordotic angulation and a caudal endplate having 9 degrees of lordotic angulation. In some instances, no lordotic correction is needed by the patient. In these situations, neutral or zero degrees of endplate angulation will be available from the implant. For example, the implant is AL-ADR or an LL-ADR and includes a cephalad endplate having 0 degrees of lordotic angulation and a caudal endplate having 0 degrees of lordotic angulation.

ii. Cephalad Endplate

The cephalad endplate, also referred to as the first endplate, may have a variety of different angulations to accommodate the differences in disc space angulation, especially at L5-S1 due to the variable sacral inclination in patients and/or to correct loss of lordosis in a patient. The cephalad endplates used in C-ADRs typically have an angulations of 0 degrees. However, for LL-ADRs and AL-ADRs, the cephalad endplates can have an angulations of about 3 degrees.

### 1. Contour of Dome Configured to Match the Contour of the Superior Surface of the Core

In some embodiments, the concavity of the interior surface of the domed portion of the cephalad endplate matches the convexity of the core, such that the superior surface of the core fits inside the dome in the manner of a ball-and-socket. In these embodiments, the superior portion of the core sits inside the domed portion. When aligned in this manner, minimal translation of the cephalad endplate is possible between flexion and extension of the patient's spine.

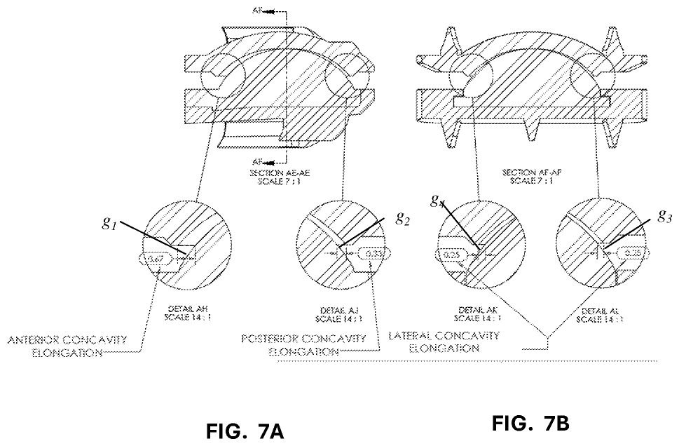

For example, looking at a cross-sectional view of the implant, the superior portion 410 (delineated from the rest of the core with line (lₛ) on FIG. 1C) of the central core 400 is recessed to a depth (d₁) into the inferior surface of the cephalad endplate. For example, the depth (d₁) of the core 400 that fits inside the domed portion 210 can be in the range of about 1 to 3 mm, optionally, about 1-2 mm, optionally about 1.5 to about 2 mm, such as about 1.5, about 1.6, about 1.7, about 1.8, about 1.9, or about 2 mm (see, e,g. FIG. 1C; see also FIG. 8B).

2. Contour of Dome Configured to be Offset from the Contour of the Superior Surface of the Core

In some instances, the concavity of the domed portion of the cephalad endplate in the implant is nonconcentric. For example, the interior surface of the domed portion can be elongated anteriorly relative to the superior surface of the core of the implant. This elongation allows the cephalad endplate, and thus the cephalad vertebrae, to translate posteriorly with extension. Optionally, the cephalad endplate in the implant is elongated posteriorly relative to the superior surface of the core of the implant, which allows the cephalad endplate, and thus the cephalad vertebrae, to translate anteriorly with extension. Further, optionally the cephalad endplate in the implant is elongated laterally on each of its sides relative to the superior surface of the core of the implant when viewed in a mid-coronal view. This lateral elongation allows the cephalad endplate, and thus the cephalad vertebrae, to translate laterally with extension.

In some instances, the diameter of the concavity in the coronal plane remains the same as that of the previously described ball-and-socket configuration described above, only the sagittal plane dimension is slightly longer by extending anteriorly and/or posteriorly. For example, the interior diameter of the dome in the sagittal plane is greater than the diameter of the core, such as at least about 1-5% greater, optionally up to about 10% greater. Having a domed portion that has a slightly elongated concavity in the sagittal plane at the anterior end of the endplate (e.g., ball-and-trough rather than the concentric ball-and-socket configuration) allows the cephalad endplate to translate posteriorly with extension of the patient's spine. Having a domed portion that has a slightly elongated concavity in the sagittal plane at the posterior end of the allows the cephalad endplate to translate anteriorly with extension of the patient's spine. Optionally, the domed portion also has a widening at each of its sides relative to the superior surface of the core, when view in the mid coronal plane, which allows the cephalad endplate, and thus the cephalad vertebrae, to translate laterally with extension.

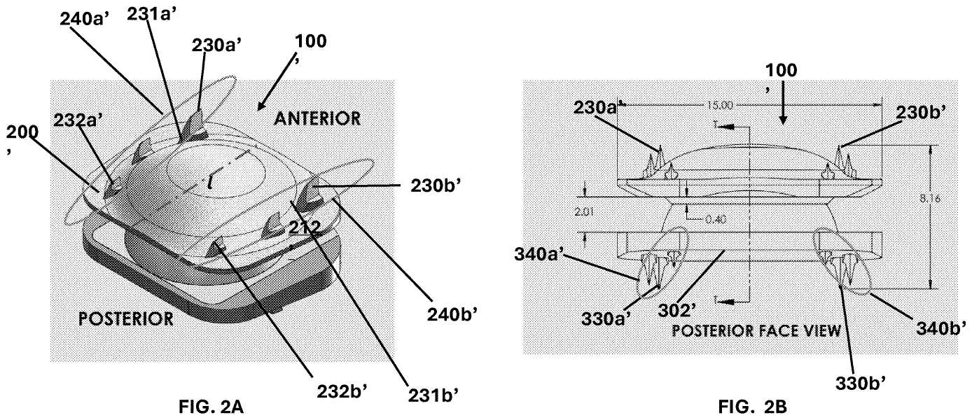

For example, as shown in the mid-sagittal view depicted in FIG. 3A, a gap (g) exists between the superior portion of the core and the undersurface of the concavity (i.e. the domed portion) of the cephalad endplate at the anterior end of the implant. In this embodiment, the concavity of the undersurface of the domed portion of the cephalad endplate is offset anteriorly from the superior portion of the core, such that a gap (g) of at least 0.1 mm, such as in the range of about 0.1 to 0.5 mm, 0.5 to 1 mm, 0.1 to 2 mm, 0.5 to 2 mm, 1 to 2 mm, exists between the anterior end of the superior portion of the core and the anterior end of the undersurface of the domed portion when the implant is in neutral alignment. The distance of the gap can be measured in a mid-sagittal view of the implant. A mid-coronal view of this ball-and-trough design shows a congruent fit between the concavity of the cephalad endplate and the core of the dome (and thus no additional lateral translation of the cephalad endplate is possible, in a similar manner as the implant having a ball-and-socket configuration).

For example, in some instances in a C-ADR, the curvature of the concavity of the undersurface of the domed portion of the cephalad endplate is elongated anteriorly by about 1 mm. This approximately 1 mm elongation allows the articulating cephalad endplate to translate farther posteriorly by about 1 mm, thereby more closely recreating the instantaneous center of rotation (ICR) of the device through the physiologic range of motion during flexion and extension (Manfred et al.).

Similarly, in some instances in an AL-ADR, the curvature of the interior surface of the domed portion of the cephalad endplate is elongated anteriorly by about 2 mm. This 2 mm elongation allows the articulating cephalad endplate to translate farther posteriorly, thereby more closely recreating the instantaneous center of rotation of the device through the physiologic range of motion during flexion and extension (Manfred et al.).

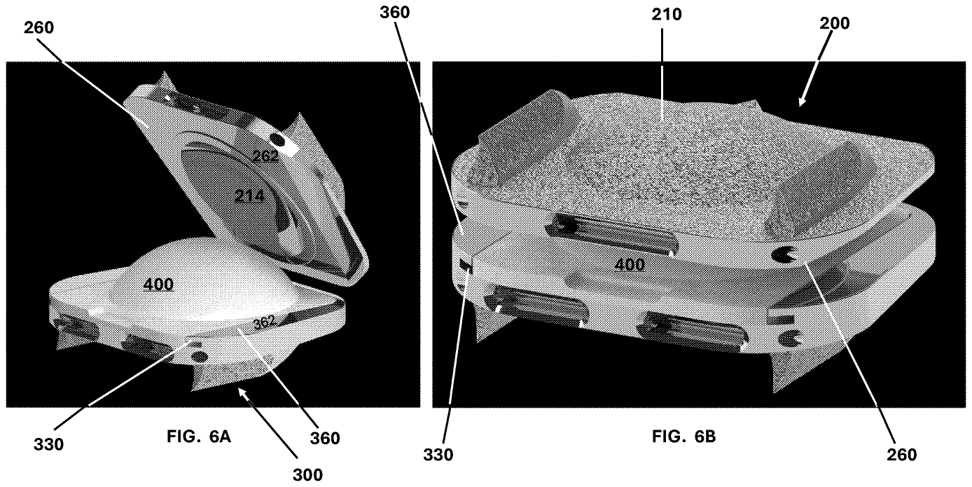

In another instance, the concavity of the inferior surface of the cephalad endplate may be offset, in a nonconcentric design, with an elongation slightly anteriorly and posteriorly from the outer anterior and posterior margins of the core, respectively; and in a similar manner, the nonconcentric design may include a slight widening on both sides of the concavity in the mid coronal plane. For example, as shown in the mid-sagittal view depicted in FIG. 7A, a gap (g₁) exists between the superior portion of the core and the undersurface of the concavity (i.e. the domed portion) of the cephalad endplate at the anterior end of the implant. In this exemplary implant, the concavity of the interior surface of the domed portion of the cephalad endplate is also offset posteriorly from the superior portion of the core, such that a gap (g₂) exists between the posterior end of the superior portion of the core and the posterior end of the inferior surface of the domed portion when the implant is in neutral alignment. The distance of the gap can be measured in a mid-sagittal view of the implant at the location with the greatest offset.

FIG. 8A is a schematic of an exemplary cervical artificial disc implant where the concavity of the domed portion of the cephalad endplate is nonconcentric relative to the outer anterior and posterior margins of the core. Although not shown in FIG. 8A, as shown in FIG. 7B, when viewed at the mid-coronal plane, the same cervical artificial disc implant is nonconcentric with the convexity of the superior surface of the core, such that the domed portion is wider on each with a side relative to the diameter of the superior portion of the core. Thus, when viewed in the mid coronal plane, each side of the domed portion has a widening concavity that can be measured where the offset is the greatest as a gap (g₃ and g₄) relative to the core. Typically, the gaps on each side of the domed portion when viewed in the mid coronal plane have the same or substantially the same size.

For example, in a cervical disc replacement implant, the anterior offset can have a gap g₁ in the range of at least of at least 0.1 mm, such as in the range of about 0.1 to 2 mm, 0.1 to 1 mm, 0.3 to 2 mm, 0.3 to 1 mm, 0.5 to 2 mm, 0.5 to 1 mm, 0.5 to 0.9 mm, 0.5 to 0.8 mm, or 0.5 to 0.7 mm, optionally 0.6 mm, 0.61 mm, 0.62 mm, 0.63 mm, 0.64 mm, 0.65 mm, 0.66 mm, 0.67 mm, 0.68 mm, 0.69 mm, or 0.7 mm. For example, in a cervical disc replacement implant, the posterior offset can have a gap g₂ in the range of at least 0.1 mm, such as in the range of about 0.1 to 0.5 mm, 0.1 to 1 mm, 0.1 to 0.4 mm, 0.1 to 0.3 mm, or 0.1 to 0.35 mm, optionally 0.3 mm, 0.31 mm, 0.32 mm, 0.33 mm, 0.34 mm, or 0.35 mm.

For example, in a lumbar disc replacement implant, the anterior offset can have a gap g₁ in the range of at least of at least 0.2 mm, such as in the range of about 0.2 to 3 mm, 0.2 to 2 mm, 0.6 to 2 mm, 0.6 to 3 mm, 1 to 3 mm, 1 to 2 mm, 1 to 1.8 mm, 1 to 1.6 mm, or 1 to 1.4 mm, optionally 1.1 mm, 1.2 mm, 1.22 mm, 1.24 mm, 1.26 mm, 1.28 mm, 1.3 mm, 1.32 mm, 1.34 mm, 1.36 mm, 1.38 mm, or 1.4 mm. For example, in a lumbar disc replacement implant, the posterior offset can have a gap g₂ in the range of at least 0.1 mm, such as in the range of about 0.1 to 2 mm, 0.1 to 1 mm, 0.1 to 0.9 mm, 0.1 to 0.8 mm, 0.1 to 0.7 mm, 0.4 to 0.7 mm, or 0.6 to 0.7 mm, optionally 0.6 mm, 0.61 mm, 0.62 mm, 0.63 mm, 0.64 mm, 0.65 mm, 0.66 mm, 0.67 mm, 0.68 mm, 0.69, or 0.7 mm.

A mid-coronal view of this exemplary implant is depicted in FIG. 7B, where the concavity of the interior surface of the domed portion of the cephalad endplate is offset laterally on both sides from the superior portion of the core, such that a gap, g₃ and g₄, exists on each side the undersurface of the domed portion relative to the of the superior portion of the core, when the implant is in neutral alignment. Optionally the lengths of gaps, g₃ and g₄, are the same or substantially the same. For example, in a cervical disc replacement implant each side, optionally both sides, of the interior surface of the domed portion can have a gap g₃ and g₄, in the range of at least 0.1 mm, such as in the range of about 0.1 to 1 mm, 0.1 to 0.5 mm, 0.1 to 0.4 mm, 0.1 to 0.3 mm, or 0.1 to 0.25 mm, measured in a mid-coronal view of the implant at the location with the greatest offset.

For example, in a lumbar disc replacement implant each side, optionally both sides, of the interior surface of the domed portion can have a gap g₃ and g₄ in the range of at least 0.2 mm, such as in the range of about 0.2 to 2 mm, 0.2 to 1 mm, 0.2 to 0.8 mm, 0.2 to 0.6 mm, 0.2 to 0.5 mm, measured in a mid-coronal view of the implant at the location with the greatest offset.

Optionally, when an exemplary cervical implant is viewed in neutral alignment, these gaps creating the offsets in the cervical disc replacement will be in the range of 0.67 mm anterior and 0.33 mm posterior at the midsagittal plane and 0.25 mm on both sides viewed in the mid coronal plane. Optionally, for an exemplary lumbar disc replacement implant, these measurements will be in the range of 1.3 mm anterior elongation of the concavity, 0.67 mm posterior elongation of the concavity in the midsagittal plane and 0.5 mm widening on each side of the concavity at the mid coronal plane.

f. Overall Dimensions of the Implant
i. Cervical ADR

In some embodiments of the cervical ADR, the configuration of the cephalad endplate allows the superior portion of the core to be recessed by a depth d into the interior surface of the domed portion. In some embodiments of the cervical ADR, the depth of the core within the dome is about 2 mm and the distance between the inferior surface of the cephalad endplate and the superior surface of the caudal endplate is about 2 mm. Thus, the posterior intervertebral disc height of the C-ADR implant can be reduced to about 4 mm. Alternative suitable posterior intervertebral disc heights of the C-ADR implant are in the range of about 4 mm to about 6 mm. Optionally, the C-ADR implant has a posterior intervertebral disc height of 4 mm, 5 mm, or 6 mm.

These smaller height implants are needed for smaller patients or in patients that preoperatively have greater intervertebral disc height collapse at the level(s) of C-ADR implantation. The configuration in which the superior portion of the core fits inside and is recessed within the interior surface of the domed portion of the cephalad endplate allows for relatively smaller height options (compared to commercially available C-ADR). Wang, et al. indicates that increasing disc height correction, beyond preoperative 5 mm height can results in a corresponding decrease in cervical range of motion (ROM) and increase in facet joint pressure. Additionally, the cumulative height restoration in multi-level C-ADR can cause the patient to have pain and/or traction radiculopathy or palsy when the overall height is relatively large, thus the use of C-ADRs with smaller intervertebral disc heights can be beneficial.

The implant can have a suitable set of dimensions with respect to height, width, depth, and lordosis to match the anatomy and needs of the patient.

Height options for the C-ADR can include 4 mm, 5 mm and 6 mm when measured at the posterior aspect of the disc space. An implant having any of the height options disclosed herein, can have a width in the range of about 15 mm to about 19 mm and a depth of about 12 mm to about 18 mm. Typical widths and their corresponding depths for C-ADR implants are listed in Table 1 below.

TABLE 1
Standard Dimensions for C-ADR Implants

| Dimension option | Width (mm) | Depth (mm) |
|---|---|---|
| 1 | 15 | 12 |
| 2 | 15 | 14 |
| 3 | 17 | 14 |
| 4 | 17 | 16 |
| 5 | 19 | 16 |
| 6 | 19 | 18 |

ii. Lumbar ADR

Similarly, in the AL-ADR and LL-ADR, the configuration of the cephalad endplate allows the superior portion of the core to be recessed by a depth dinto the interior surface of the dome. In some embodiments, the depth of the core within the dome is about 1 to 2 mm and the distance between the undersurface of the cephalad endplate and the superior surface of the caudal endplate is about 4 mm. Thus, the posterior intervertebral disc height of the AL-ADR or LL-ADR implant is about 6 mm. Alternative suitable posterior intervertebral disc heights of the AL-ADR or LL-ADR implant are in the range of about 6 mm to about 12 mm. Optionally, the AL-ADR or LL-ADR implant has a posterior intervertebral disc height of about 6 mm, 8 mm, 10 mm, or 12 mm.

The implant can have a suitable set of dimensions with respect to height, width, depth, and lordosis to match the anatomy and needs of the patient.

Height options for the AL-ADR or LL-ADR can include 6 mm, 8 mm, 10 mm, and 12 mm when measured at the posterior aspect of the disc space. An implant having any of the height options disclosed herein, can have a width in the range of about 15 mm to about 19 mm and a depth of about 12 mm to about 18 mm. Typical widths and their corresponding depths for AL-ADR or LL-ADR implants are listed in Table 1 above.

### 3. Fixation Elements

The first and second endplates include one or more fixation elements configured to engage the adjacent vertebral bodies, thereby fix the implant to and retain the implant in the desired location between the adjacent vertebral bodies following implantation. Exemplary fixation elements include spikes and keels, with a cutting edge pointing towards the posterior end of the implant. Additionally, the bone-contacting surface of either or both endplates may contain a rough surface, such as in the form of knurls, ridges, spikes, or similar small projections to aid in securing the implant to the vertebral body and prevent or reduce shifting of the implant. The surface roughness can also be formed by the porosity of the surface coating on the bone-contacting surface of the endplate.

a. Shape and Dimensions

The caudal and/or cephalad endplates may contain one or more keels, optionally at least two keels or more keels, that have the same or different geometries and sizes. An implant containing endplates with exemplary keels is illustrated in FIGS. 1A-1E. As shown in FIG. 1A, each keel includes two side portions and a tapered, sharp end (“the posterior edge” of the keel) pointing toward the posterior end of the endplate.

The two side portions of each keel meet along their length at a sharp edge that is adjacent to and contacts the proximal vertebral body when the implant is inserted in a patient. The edge of the keels may angulate 0° to 20° laterally, optionally the edge of each keel angulates 0° laterally, about 0.5° to 5° laterally, about 0.5° to about 3°, about 1 to about about 5° to 10° laterally, about 10° to 15° laterally, or about 15° to 20° laterally, or optionally about 15° laterally.

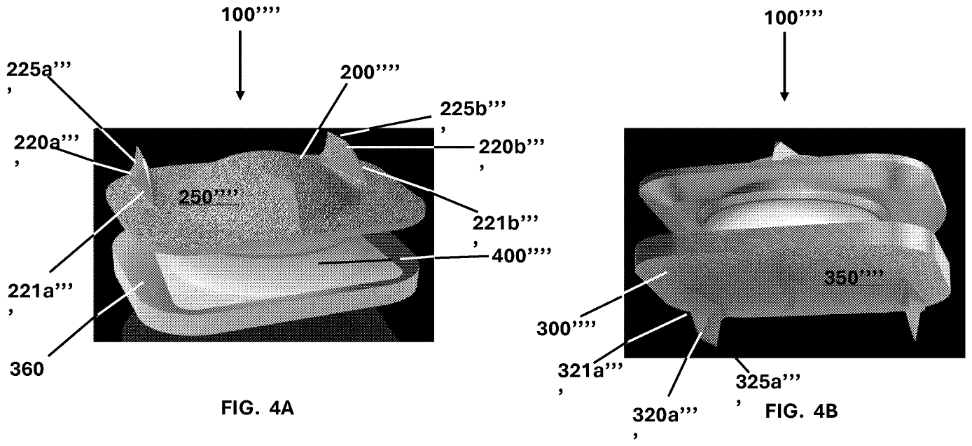

The shape of the keels and/or spikes widens towards the anterior end of the implant to provide a wedge or press fit into the bone endplate. Further, when viewed laterally (see, e.g. FIG. 5B), the leading edge posterior edge, e.g. 221b″″, 221a″″, 321b″″ (i.e. the posterior aspect of the keel and/or spike) curves away from the spinal canal, i.e. curving anteriorly from the base of the endplate side to the tip 222b″′, 322b″′ of the sharp leading posterior edge., (FIG. 5B). In some instances, the fixation elements have a suitable shape to improve the safety of the spinal disc replacement surgery. For example, the fixation elements do not have a flat-front, such as observed in the existing prodisc® C implant (Centinel Spine) (see e.g., FIG. 5A), which could potentially cause a vertebral fracture which could retropulse into the spinal canal. This is useful for patients with advanced spondylosis and bone spur formation along the posterior vertebral margin.

Further, as shown in FIG. 5B, the implant described herein has the center of rotation of the core positioned slightly posterior to the mid-portion of the endplates in the sagittal plane.

In some instances, the anterior portion of both the keel and/or spike is wider than the posterior edge, with the keel tapering from the wide anterior end to the narrower posterior edge, such as in the form of a wedge, to create a press fit into the host bone. These keels and/or spikes are tapered such that the smooth leading posterior edge is narrower than the anterior end, for example the leading posterior edge may be 10% to 100%, 10% to 20%, 10% to 30%, 20% to 40%, 20% to 30%, 30% to 40%, 30% to 50%, 40% to 60%, 40% to 50%, 50% to 70%, 50% to 60%, 60% to 80%, 60% to 70%, 70% to 90%, 70% to 100%, 70% to 80%, 70% to 90%, 80% to 100%, or 80% to 90% narrower than the width of the anterior end. For example, the leading posterior edge of the keel and/or the spike can have a width in the range of 0.1 to 1.5 mm, such as in the range of 0.1 mm to 0.5 mm, 0.3 mm to 0.8 mm, 0.5 mm to 0.9 mm, 0.2 mm to 0.6 mm, 0.4 mm to 0.7 mm, 0.6 mm to 1.0 mm, 0.8 mm to 1.2 mm, 0.9 mm to 1.3 mm, 1.0 mm to 1.4 mm, 1.1 mm to 1.5 mm. The anterior end of the keel and/or the spike can have a width in the range of about 1 to 2 mm, such as in the range of 1 mm to 1.2 mm, 1.1 mm to 1.5 mm, 1.3 mm to 1.8 mm, 1.2 mm to 1.7 mm, 1.4 mm to 1.9 mm, 1.5 mm to 2.0 mm, 1.0 mm to 1.4 mm, 1.6 mm to 2 mm, 1.3 mm to 1.6 mm, or 1.7 mm to 2 mm.

For example, in some instance the width of the leading posterior edge of the keel and/or spike is 1 mm and the width of the keel or spike increases to the anterior end with a width of about 1.5 mm. Optionally the keel and/or spike or portions, such as the side portions thereof located towards the anterior of the endplate, include the same surface coating as the surfaces of the endplate effacing the host bone to aid in implant fixation.

b. Posterior Edge Sufficiently Sharp that an Initial Chiseling Step is not Needed Prior to Insertion

Optionally the posterior edge of the one or more keels is sufficiently sharp to cut into the adjacent vertebral body as it is inserted between the vertebrae (see, e.g. FIGS. 4A and 4B). Optionally, the side surfaces of the keel, particularly in the regions toward the anterior end of the endplate, have a textured surface, such as a porous surface configured to promote bone ingrowth and attachment. Optionally, the posterior edge of the keel is sufficiently sharp to allow for the insertion of the implant in the absence of an initial milling or chiseling step to mill or chisel into the surfaces of the superior and inferior vertebral bodies prior to insertion of the implant to facilitate insertion of the implant.

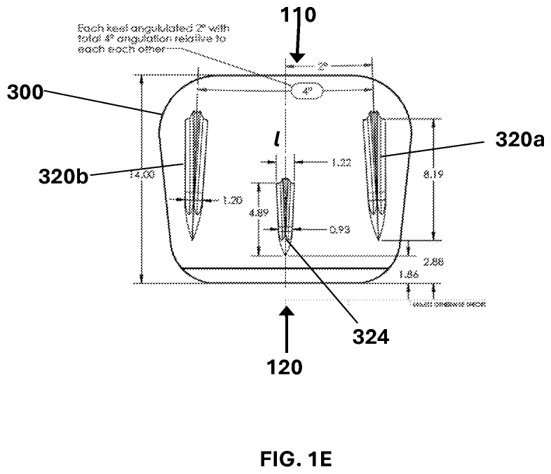

The caudal and/or cephalad endplates may contain one or more spikes, optionally at least two keels or more spikes, that have the same or different geometries and sizes. An implant containing endplates with exemplary spikes 230a′, 230b′ is illustrated in FIGS. 2A and 2B. As shown in FIG. 2A, each spike includes a tapered, sharp end (“the posterior edge” of the spike) 231a′ 231b′ pointing toward the posterior end of the endplate.

Optionally the posterior edge of the one or more spikes is sufficiently sharp to cut into the adjacent vertebral body as it is inserted between the vertebrae (see, e.g. FIG. 2A). Optionally, the side surfaces of the spike, particularly in the regions toward the anterior end of the endplate, have a textured surface, such as a porous surface configured to promote bone ingrowth and attachment. Optionally, the posterior edge of the spike(s) is sufficiently sharp to allow for the insertion of the implant in the absence of an initial milling or chiseling step to mill or chisel into the surfaces of the superior and inferior vertebral bodies prior to insertion of the implant to facilitate insertion of the implant.

c. Exemplary Arrangement of Fixation Elements on Cephalad and Caudal Endplates

In some instances, the cephalad endplate includes two keels that are aligned parallel to each other, where each keel is on top of and attached to the flange, and a first keel is located on one side of the domed portion and the second keel is located on the opposite side of the endplate. In some instances, the cephalad endplate includes two keels that are aligned parallel to each other, where each keel is located on top of and/or intersects with a region of the domed portion of the endplate. Optionally the cephalad endplate also includes one smaller keel in the posterior end of the endplate, such as located in the center adjacent to the domed portion of the endplate (see FIG. 4A). The caudal endplate can have one or more keels. Optionally, the caudal endplate contains three keels, with one keel on each side of the caudal endplate and located towards the anterior end of the endplate, and a third keel located at or towards the posterior end of the endplate and optionally in the center of the endplate, such as illustrated in FIG. 4B. The side keels can be located closer to each other or farther apart on the caudal endplate, relative to how they are illustrated in FIG. 4B.

In some instances, the cephalad endplate includes two keels that are aligned such that each keel is on an opposite side of the domed portion and angle laterally relative to a center line (l) that runs from the posterior portion 120 of the endplate to the anterior portion 110 of the endplate, where each keel is on top of and attached to the flange, and a first keel is located on one side of the domed portion and the second keel is located on the opposite side of the endplate. Optionally, the cephalad endplate includes a third smaller keel in the center of the endplate. For example, FIG. 1E shows a caudal endplate 300, containing two side keels 320a, 320b that have a substantially symmetric orientation relative to each other about the center line (l) (line of symmetry) and a smaller central keel 324 located toward the posterior end 120 of the endplate and in substantial alignment with the center line (l). The side keels are oriented slightly divergent from the anterior end to posterior end relative to each other. This creates a press fit from the two side keels following insertion into the adjacent vertebrae. The angulation of each side keel relative to the center line (l) can range from 0.1° to 20°, 0.1° to 10°, 0.1° to 5°, 0.1° to 3°, 0.1° to 2.5°, 0.1° to 2°, 0.5° to 20°, 0.5° to 10°, 0.5° to 5° 0.5° to 3°, 0.5° to 2.5°, 0.5° to 2°, 0.5° to 1°, 1° to 20°, 1° to 10°, 1° to 5°, 1° to 3°, 1° to 2.5°, or 1° to 2°. Optionally the total divergence in the angulation of the side keels relative to each other ranges from 0.2° to 40°, 0.2° to 20°, 0.2° to 10°, 0.2° to 6°, 0.2° to 5°, 0.2° to 4°, 1° to 40°, 1 to 20°, 1° to 10°, 1° to 6°, 1° to 5° 1° to 4° 1° to 2°, 2° to 40°, 2° to 20°, 2° to 10°, 2° to 6°, 2° to 5°, or 2° to 4°.

While FIG. 1E illustrates the angulation of the two side keels on a caudal endplate, the side keels and/or groups of spikes on the corresponding cephalad endplate can also have divergent keel orientation, where the side keels and/or groups of spikes of the cephalad endplate are symmetric relative to each other about a center line and can have the same or different angulations than the angulation of the side keels and/or groups of spikes on the corresponding caudal endplate in the same implant. The orientation of two groups of spikes relative to a center line (l) running anteriorly and posteriorly along an exemplary cephalad endplate is illustrated in FIG. 2A.

### B. Methods of Making

In some instances, the endplates can be formed in a single step using 3-dimensional additive manufacturing techniques. For example, in embodiments in which the endplates are formed from materials such as a titanium-ceramic polymer, e.g. Ti-6Al-4V, or commercially pure titanium, the same material that is used to produce the endplate itself is used to form the surface coating/texture to promote bone ingrowth or ongrowth. Thus, this is a continuous process, and no secondary step or bonding step is involved in this process to add a porous surface coating to the exterior surface(s) of the endplates. The use of the same material when forming the coating as the material that is used to form the endplate results in excellent bonding between the surface coating and the endplate and thereby reduces or prevents delamination of the surface coating from the endplate.

In other instances, a two-step process is needed. For example, in embodiments in which the endplates are formed from non-metal materials, such as biocompatible polymers, e.g. polyether-ether-ketone (PEEK), carbon-fiber polyether-ether-ketone (CF-PEEK) or a ceramic alloy, after the endplate is formed, such as via 3-dimensional additive manufacturing, a second step is used to apply and bond a plasma spray titanium surface coating to the exterior surface(s) of the endplates. In other instances, any of the material options for the endplates can be coated in the articulating concavity with TiN (titanium nitride) to reduce friction and improve wear resistance between the core and the corresponding articulating surface on the cephalad or caudal endplate.

Exemplary combinations for endplate materials along with their general manufacturing process and core materials are summarized in Table 2.

TABLE 2
Exemplary combinations of Materials for Implants

| | Articulating Core |
|---|---|
| Endplate Materials and Manufacturing Process | Materials |
| 1) Titanium Polymer (+titanium polymer additive | a) UHMWPE |
| manufacturing of surface coating). One step | b) Ceramic Polymer |
| manufacturing. | |
| 2) Commercially pure titanium (+commercially pure | a) UHMWPE |
| additive manufacturing of surface coating). | b) Ceramic Polymer |
| One step manufacturing. | |
| 3) CF/PEEK (+traditional plasma spray titanium | a) Ceramic polymer |
| endplate coating). Two step manufacturing. | |
| 4) Ceramic Alloy (+traditional plasma spray | a) UHMWPE |
| titanium endplate coating). Two step manufacturing. | b) Ceramic Polymer |

### C. Insertion Methods

The assembly of the caudal endplate, cephalad endplate, and core constitutes the total C-ADR (see, e,g. FIG. 1A), LL-ADR, or AL-ADR to be implanted. This allows for a one-step insertion of a fully assembled implant.

The implant is typically provided in a pre-assembled form, where the inferior surface of the core is inserted into and fixedly secured to the caudal endplate, such as within a depression in the interior surface of the caudal endplate. The superior portion of the core fits inside the domed portion of the cephalad endplate.

If the patient's anatomy is such that the inferior endplate of the cephalad vertebral body is flat and does not form fit to the domed portion of the cephalad endplate, the method includes the step of milling the disc space, prior to insertion of the implant in the disc space, so that it is more concave and conforms to the domed portion of the cephalad endplate.

The preassembled implant is inserted such that the cephalad endplate and the opposing core fixed to the caudal endplate are implanted as a whole, allowing for a single-step insertion.

The fixation elements on the exterior surface of each of the endplates have a sufficiently sharp posterior edge to cut into the adjacent vertebral body as the implant is inserted between the vertebrae. Thus, an initial chiseling step is not needed prior to insertion of the implant.

After the damaged disc is removed from between adjacent first and second vertebral bodies (i.e. adjacent cephalad and caudal vertebral bodies), typically a trial implant is used to ensure that the correctly sized implant is selected for the disc space. The trial is removed and an implant described herein having a suitable size for the disc space is inserted into the disc space. The fixation elements on the exterior surface of each of the endplates have a sufficiently sharp posterior edge to cut into the adjacent vertebral body as the implant is inserted between the vertebrae. The method generally does not involve an initial chiseling or drilling step to prepare the surfaces of the vertebral bodies prior to inserting the implant therebetween.

### D. Kits

Separate kits for each of C-ADR, LL-ADR and AL-ADR are provided. Each kit can include insertion and removal instruments, two or more trial implants to measure height, width and depth of implant needed at the site of insertion in the spine for the patient in need thereof. Optionally, the kit includes a suitable tool for milling the disc space when needed.

Optionally the kit includes one or more insertion instruments or attachment portions, which are optionally registered such that robotic navigation can be employed to improve accuracy of midline placement and a tangential insertion of the implants disclosed herein.

Optionally the kit includes one or more additional instruments, such as a distractors for the disc space, and/or an intervertebral spreader.

### 1. Cervical

In some instances, the kit includes more than one trial implant that can be used to select a cervical artificial disc (C-ADR) that has suitable dimensions for the patient. Each trial implant has a different set of dimensions with respect to height, width, depth, and lordosis.

Height options for the C-ADR can include 4 mm, 5 mm and 6 mm when measured at the posterior aspect of the disc space. Each trial implant height option in the kit is provided in one or more, optionally more than one width and depth, such as for example in one or more, optionally all, of the widths and depths listed in Table 1 above.

### 2. Lumbar

In some instances, the kit includes more than one trial implant that can be used to select an AL-ADR or an LL-ADR that has suitable dimensions for the patient. Each trial implant has a different set of dimensions with respect to height, width, depth, and lordosis.

Height options for the AL-ADR and the LL-ADR can include 6 mm, 8 mm, 10 mm, and 12 mm when measured at the posterior aspect of the disc space. Each trial implant height option in the kit is provided in one or more, optionally more than one width and depth, such as for example in one or more, optionally all, of the widths and depths listed in Table 1 above.

The disclosed kits can also include one or more tools for aiding in the insertion of the implant and/or instructions for use.

The components described above as well as other components can be packaged together in any suitable combination as a kit useful for performing, or aiding in the performance of, the disclosed method.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

The disclosed implants, as well as methods of making and using thereof, can be further understood by reference to the following numbered paragraphs:
Paragraph 1. An artificial disc replacement implant comprising a cephalad endplate, a caudal endplate, and a core, wherein the core is disposed between the cephalad endplate and the caudal endplate,
   wherein the cephalad endplate comprises a domed portion,
   wherein the superior portion of the core has a convex surface configured to fit inside the domed portion of the cephalad endplate, and
   wherein the superior surface of the cephalad endplate and the inferior surface of the caudal endplate comprise one or more textured or porous regions to facilitate bone ongrowth and/or ingrowth and one or more fixation elements.
Paragraph 2. The implant of Paragraph 1 configured for insertion between two vertebrae in the cervical or lumbar region of the spine in a patient.
Paragraph 3. The implant of Paragraph 1 or 2, wherein the caudal endplate has an anterior end and a posterior end, and wherein the anterior end has a height that is greater than the height of the posterior end, optionally wherein following insertion into the patient's spine a desired degree of lordosis is obtained.
Paragraph 4. The implant of any one of Paragraphs 1 to 3, wherein caudal endplate has a lordotic angulation of about 0 degrees to about 9 degrees, from about 3 degrees to about 9 degrees, from about 3 degrees to about 6 degrees, or from about 6 degrees to about 9 degrees, optionally wherein the lordotic angulation is about 0 degrees, about 3 degrees, about 6 degrees, or about 9 degrees.
Paragraph 5. The implant of any one of Paragraphs 1 to 4, wherein the one or more fixation elements are keels, spikes, or a combination thereof, wherein the one or more fixation elements comprise a sharp leading edge at the posterior aspect of the fixation element, and widen toward the anterior end of the implant, optionally wherein the leading edge at the posterior aspect of at least one of the fixation elements curves away from the posterior end toward the anterior end of the implant.
Paragraph 6. The implant of any of Paragraphs 1 to 5, wherein the fixation elements are two or more keels, optionally wherein the cephalad endplate comprises a first keel on a first side of the domed portion and a second keel on the opposite side of the domed portion, wherein the first and the second keels are symmetrically aligned relative to each other about a center line of symmetry (l) that runs anteriorly and posteriorly, and optionally a third keel, wherein the third keel is located adjacent to the posterior end of the cephalad endplate, and wherein the third keel is smaller that each of the first and second keels; and
   optionally wherein the caudal endplate comprises a fourth keel and a fifth keel, wherein the fourth and fifth keels are symmetrically aligned relative to each other about a center line of symmetry (l) that runs anteriorly and posteriorly, and are located on opposite sides of the exterior surface and optionally a sixth keel, wherein the sixth keel is located adjacent to the posterior end of the cephalad endplate, and wherein the sixth keel is smaller that each of the fourth and fifth keels.
Paragraph 7. The implant of Paragraph 6, wherein the first and second keels are aligned so that they diverge when viewed from the posterior end of the endplate to the anterior end of the endplate.
Paragraph 8. The implant of Paragraph 7, wherein the angulation of each of the first and second keels relative to the center line (l) can range from 0.1° to 20°, 0.1° to 10°, 0.1° to 5°, 0.1° to 3°, 0.1° to 2.5°, 0.1° to 2°, 0.5° to 20°, 0.5° to 10°, 0.5° to 5° 0.5° to 3° 0.5° to 2.5°, 0.50 to 2°, 0.50 to 1°, 10 to 20°, 10 to 10°, 10 to 5° 10 to 3°, 1° to 2.5°, or 1° to 2°.
Paragraph 9. The implant of any one of Paragraphs 1 to 5, wherein the fixation elements are two or more spikes, optionally wherein the cephalad endplate comprises a first group of spikes on a first side of the domed portion and a second group of spikes on the opposite side of the domed portion, wherein the first and the second groups of spikes are substantially symmetrical, and wherein each group of spikes contains at least two spikes, optionally having different sizes, with the largest spike is located at the anterior end of the cephalad endplate; and
   optionally wherein the caudal endplate comprises a third group of spikes and a fourth group of spikes, wherein the third and fourth groups of spikes are substantially symmetrical, and are located on opposite sides of the exterior surface, and wherein each group of spikes contains at least two spikes, optionally having different sizes, with the largest spike is located at the anterior end of the cephalad endplate.
Paragraph 10. The implant of any one of Paragraphs 1 to 9, wherein the concavity of the inferior surface of the domed portion of the cephalad endplate matches the convexity of the superior portion of the core, such that the core fits inside the concavity in a ball-and-socket configuration.
Paragraph 11. The implant of any one of Paragraphs 1 to 9, wherein the concavity of the inferior surface of the domed portion of the cephalad endplate is nonconcentric relative to the convexity of the superior portion of the core, such that the domed portion of the inferior surface of the cephalad endplate is elongated anteriorly and optionally also elongated posteriorly relative to the core, optionally each side of the domed portion of the inferior surface of the cephalad endplate is wider than the superior portion of the core when viewed in the mid coronal plane.
Paragraph 12. The implant of Paragraph 11, wherein when the implant is in neutral alignment and is viewed from the mid sagittal view, the anterior elongation of the inferior surface of the domed portion relative to the superior portion of the core has a first gap (g₁),
   optionally the posterior elongation of the inferior surface of the domed portion relative to the superior portion of the core has a second gap (g₂), and
   optionally when the implant in neutral alignment and is viewed in the mid coronal plane, the domed portion is offset laterally on both sides from the superior portion of the core, such that a third and fourth gap, g₃ and g₄, exist on each side the inferior surface of the domed portion relative to the of the superior portion of the core.
Paragraph 13. The implant of Paragraph 12, wherein the implant is a cervical disc replacement implant, and wherein the first gap (g₁) has a length of at least 0.1 mm, optionally in the range of 0.1 to 2 mm, 0.1 to 1 mm, 0.3 to 2 mm, 0.3 to 1 mm, 0.5 to 2 mm, 0.5 to 1 mm, 0.5 to 0.9 mm, 0.5 to 0.8 mm, or 0.5 to 0.7 mm, optionally 0.6 mm, 0.61 mm, 0.62 mm, 0.63 mm, 0.64 mm, 0.65 mm, 0.66 mm, 0.67 mm, 0.68 mm, 0.69 mm, or 0.7 mm.
Paragraph 14. The implant of Paragraph 12 or 13, wherein the implant is a cervical disc replacement implant, and wherein the second gap (g₂) is present and has a length of at least 0.1 mm, optionally in the range of about 0.1 to 0.5 mm, 0.1 to 1 mm, 0.1 to 0.4 mm, 0.1 to 0.3 mm, or 0.1 to 0.35 mm, optionally 0.3 mm, 0.31 mm, 0.32 mm, 0.33 mm, 0.34 mm, or 0.35 mm.
Paragraph 15. The implant of any one of Paragraphs 12 to 14, wherein the implant is a cervical disc replacement implant, and wherein the third and fourth gaps (g₃ and g₄) are present and independently each of the third and fourth gaps has a length of at least 0.1 mm, optionally in the range of about 0.1 to 1 mm, 0.1 to 0.5 mm, 0.1 to 0.4 mm, 0.1 to 0.3 mm, or 0.1 to 0.25 mm, optionally wherein the lengths of the third and fourth gaps are the same or substantially the same.
Paragraph 16. The implant of Paragraph 12, wherein the implant is a lumbar disc replacement implant, and wherein the first gap (g₁) has a length of at least 0.2 mm, optionally in the range of about 0.2 to 3 mm, 0.2 to 2 mm, 0.6 to 2 mm, 0.6 to 3 mm, 1 to 3 mm, 1 to 2 mm, 1 to 1.8 mm, 1 to 1.6 mm, or 1 to 1.4 mm, optionally 1.1 mm, 1.2 mm, 1.22 mm, 1.24 mm, 1.26 mm, 1.28 mm, 1.3 mm, 1.32 mm, 1.34 mm, 1.36 mm, 1.38 mm, or 1.4 mm.
Paragraph 17. The implant Paragraph 12 or 16, wherein the implant is a wherein the implant is a lumbar disc replacement implant, and wherein the second gap (g₂) is present and has a length of at least 0.1 mm, optionally in the range of about 0.1 to 2 mm, 0.1 to 1 mm, 0.1 to 0.9 mm, 0.1 to 0.8 mm, 0.1 to 0.7 mm, 0.4 to 0.7 mm, or 0.6 to 0.7 mm, optionally 0.6 mm, 0.61 mm, 0.62 mm, 0.63 mm, 0.64 mm, 0.65 mm, 0.66 mm, 0.67 mm, 0.68 mm, 0.69, or 0.7 mm.
Paragraph 18. The implant of any one of Paragraphs 12 or 16 to 17, wherein the implant is a lumbar disc replacement implant, and wherein the third and fourth gaps (g₃ and g₄) are present and independently each of the third and fourth gaps has a length of at least 0.2 mm, optionally in the range of about 0.2 to 2 mm, 0.2 to 1 mm, 0.2 to 0.8 mm, 0.2 to 0.6 mm, or 0.2 to 0.5 mm, optionally wherein the lengths of the third and fourth gaps are the same or substantially the same.
Paragraph 19. A method for replacing a damaged cervical or lumbar disc in a patient comprising:
   after the damaged disc is removed from between a first and second vertebral body, inserting the implant of any one of Paragraphs 1 to 18 into the space between the first and second vertebral bodies.
Paragraph 20. The method of Paragraph 19, wherein the method does not include a chiseling and/or drilling step to prepare the surfaces of the vertebral bodies prior to inserting the implant therebetween.

### Examples

### Example 1: Exemplary C-ADR with Keels as Fixation Elements

An exemplary C-ADR implant with keels is illustrated in FIGS. 1A-1E. As shown in FIG. 1A, implant 100 includes a cephalad endplate 200; a caudal endplate 300, and a central core 400 disposed between the two endplates. Each implant includes an anterior end 110 and a posterior end 120. Each endplate includes an exterior, i.e. bone-facing, surface and an interior, i.e. core-facing, surface. The interior surface (also referred to as the superior surface) of the caudal endplate includes a depression 330 having a suitable size and shape to receive the inferior surface 420 of the core 400.

The caudal endplate includes at least two keels 320a, 320b, optionally having the same size and shape or symmetrical shapes, on its exterior surface (also referred to as the inferior surface), where a first keel 320a is located along a first side of the inferior surface and a second keel 320b is located on the opposite side of the inferior surface. The first and second keels are symmetrically aligned about the center line (l). Optionally, the first and second keels are aligned parallel to each other and the center line. Alternatively, the first and second keels are aligned so that they diverge when view from the posterior end of the endplate to the anterior end of the endplate. The angulation of each of the first and second keels relative to the center line (l) can range from 0.1° to 20°, 0.10° to 10°, 0.1° to 5°, 0.1° to 3°, 0.1° to 2.5°, 0.1° to 2°, 0.5° to 20°, 0.5° to 10°, 0.5° to 5°, 0.5° to 3°, 0.5° to 2.5°, 0.5° to 2°, 0.5° to 1°, 1° to 20°, 1° to 10°, 1° to 5°, 1° to 3°, 1° to 2.5°, or 1° to 2°. The caudal endplate also includes a third keel located in the center of the inferior surface and terminating at or near the posterior end of the implant. The third keel can have a size and shape that is the same as the first and/or the second keel, optionally the third keel has the same length and height as the first and/or the second keel. Optionally, the third keel 324 is smaller than the first and/or the second keel 320a, 320b.

The exterior surface (also referred to as the superior surface) of the cephalad endplate includes a domed portion 210 located in or about the center of the endplate. The domed portion is surrounded by a substantially flat flange 212. The domed portion 210 has a suitable size and concavity to receive the superior portion 410 of the core 400, such that the superior portion 410 contacts the undersurface of the domed portion and fits inside the domed portion.

The cephalad endplate also includes at least two keels 220a, 220b, optionally having the same size and shape or symmetrical shapes, on its exterior surface, where a first keel is located along a first side of the superior surface and a second keel is located on the opposite side of the superior surface. The first and second keels are substantially symmetrically aligned so that their orientation mirrors each other relative to a center line (l) that runs anterioirly and posteriorly. Optionally, the first and second keels diverge when viewed from the posterior end of the endplate to the anterior end of the endplate. The angulation of each of the first and second keels relative to the center line (l) can range from 0.1° to 20°, 0.1° to 10°, 0.1° to 5°, 0.1° to 3°, 0.1° to 2.5°, 0.1° to 2°, 0.5° to 20°, 0.5° to 10°, 0.5° to 5°, 0.5° to 3°, 0.5° to 2.5°, 0.5° to 2°, 0.5° to 1°, 1° to 20°, 1° to 10°, 1° to 5°, 1° to 3°, 1° to 2.5°, or 1° to 2°. Optionally, the first and second keels are attached to and protrude from the flange 212 of the endplate, with one keel 220a on one side of the domed portion 210 and the other keel 220b on the opposite side. The cephalad endplate also includes a third keel 224 located in the center of the superior surface and terminating at or near the posterior end of the implant. The third keel can have a size and shape that is the same as the first and/or the second keel, optionally the third keel has the same length and height as the first and/or the second keel. Optionally, as shown in FIG. 1A, the third keel 224 is smaller than the first and/or the second keel 220a, 220b.

FIG. 1A shows the keels as being on top of the flange and located adjacent to the domed portion, with each keel on opposite sides of the domed portion. However, the keels may be located closer to each other, and optionally may be located on the domed portion, where a first keel is on a first region of the domed portion and the second keel is on a second region of the domed portion.

Although not shown on FIGS. 1A, 1B, and 1D, the exterior surfaces of the endplates can be textured or porous, as shown in FIGS. 4A and 4B, to promote attachment to and integration with the endplates of the adjacent vertebral bodies by facilitating bone ongrowth and ingrowth.

### Example 2. Exemplary C-ADR with Spikes as Fixation Elements

An exemplary C-ADR implant with spikes as fixation elements is illustrated in FIGS. 2A-2B. This implant has the same components and can have the same overall structure and shape as described above with respect to Example 1. However, in place of keels, the fixation elements are one or more spikes.

For example, as shown in FIG. 2A, the cephalad endplate 200′ includes two or more spikes 230a′, 230b′, 232a′, 232b′, which can have substantially the same shape and size or different shapes and/or sizes. Similarly, the caudal endplate 300′ includes two or more spikes 330a′, 330b′, which can have substantially the same shape and size or different shapes and/or sizes.

Optionally, in the cephalad endplate 200′, the spikes are attached to and protrude from the flange 212′ with a first group 240a′ of spikes located on one side of the domed portion and a second group of spikes 240b′ located on the opposite side of the domed portion. Each group of spikes contains two or more spikes on the same side or region of the endplate. Optionally each of the spikes in the first group has a matching spike in the second group of spikes. For example, the largest spike 230a′, 230b′ in each group 240a′, 240b′ can be located near the anterior end of the cephalad endplate and the smallest spike 232a′, 232b′ in each group can be located at or near the posterior end of the cephalad endplate. Similarly, in the caudal endplate 300′, optionally, the spikes are attached to and protrude from the anterior surface 302′ with a first group 340a′ of spikes located on one side of the anterior surface and a second group of spikes 340b′ located on the opposite side of the anterior surface.

The spikes in each group of spikes generally align with one spike behind the other in a substantially linear alignment. A first group of spikes can be located on one side of the surface of an endplate and a second group of spikes can be located on the opposite side of the surface, where the alignment of the first group of spikes is substantially symmetric with the alignment of the second group of spikes about a center line (l) that runs anteriorly and posteriorly along the surface of the endplate (see, e.g. FIG. 2A).

As shown in FIG. 2A, each spike includes a tapered, sharp end (“the posterior edge” of the spike) 231a′ 231b′ pointing toward the posterior end of the endplate. The posterior edge is sufficiently sharp to cut into the endplate of the adjacent vertebral body as the implant is inserted between the vertebrae. Thus, an initial chiseling step is not needed to prepare the vertebral endplate prior to insertion of the implant.

Although not shown on FIGS. 2A-2B, the exterior surfaces of the endplates can be textured or porous, as shown in FIGS. 4A and 4B, to promote attachment to and integration with the adjacent vertebra bodies by facilitating bone ongrowth and ingrowth.

### Example 3: Exemplary AL-ADR with Keels as Fixation Elements and Porous Surface on Cephalad and Caudal Endplates

An exemplary AL-ADR implant with keels is illustrated in FIGS. 4A and 4B. As shown in FIGS. 4A and 4B implant 100″″ includes a cephalad endplate 200″″; a caudal endplate 300″″, and a central core 400″′ disposed between the two endplates.

Some of all of the bone-facing surfaces of each endplate, i.e. the superior surface of the cephalad endplate (shown in FIG. 4A) and the inferior surface of the caudal endplate (shown in FIG. 4B) have a porous or textured surface 250″″, 350″″ to facilitate initial implant fixation and/or bone ongrowth and ingrowth.

Each keel 220a″″, 220b″″, 320a″″ includes a tapered, sharp end (“the posterior edge” of the keel) pointing toward the posterior end of the endplate. The keels widen towards the anterior end of the implant to provide a wedge or press fit into the bone endplate.

As shown in FIGS. 4A and 4B, the keels 220a″″, 220b″″ of the cephalad endplate 200″″ include a sharp, smooth posterior edge 221a″″, 221b″″ and the side portions 225a″″, 225b″″ located towards the anterior end of the implant are porous or textured. Similarly, the keels 320a′″ of the caudal endplate 300″″ include a sharp, smooth posterior edge 321a″″ and the side portions 325a″″ located towards the anterior end of the implant are porous or textured. For example, the side portions of the keels include the same surface coating as the surfaces of the endplate effacing the host bone to aid in implant fixation and/or promote attachment to and integration with the adjacent vertebral bodies.

The textured surface may comprise a series of small protrusions, indentations, or irregular features visible on the top face of the endplate. This textured region may cover all or a portion of the exterior surface that contacts the vertebral body.

The interior surface, i.e. core contacting surface, of each endplate 200″′, 300″″ may be substantially smooth, and the interior surfaces of the caudal and cephalad endplates are optionally coated with a titanium nitride (TiN) coating (see, e.g. FIGS. 6A and 6B).

It is understood that the disclosed implants, kits and methods are not limited to the particular embodiment described as these can vary. It is also to be understood that the terminology used herein is for the purpose of describing particular embodiments only, and is not intended to limit the scope of the present invention which will be limited only by the appended claims.

The singular forms “a,” “an,” and “the” include plural reference unless the context clearly dictates otherwise. Thus, for example, reference to “a keel” includes a plurality of such keels, reference to “the spike” is a reference to one or more spikes and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Although the description of materials, components, steps, techniques, etc. can include numerous options and alternatives, this should not be construed as, and is not an admission that, such options and alternatives are equivalent to each other or, in particular, are obvious alternatives.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Ebru Oral & Orhun K. Muratoglu. International Orthopaedics (SICOT) (2011) 35:215-223. “Vitamin E diffused, highly crosslinked UHMWPE: a review”.
2. F. Brennan Torstrick, MSa, Brett S. Klosterhoff, BSa, L. Erik Westerlund, MDb, Kevin T. Foley, MDc,d, Joanna Gochuico, BSa,e, Christopher S.D. Lee, PhDe, Ken Gall, PhDe,f,g. The Spine Journal 18 (2018) 857-865. “Impaction durability of porous polyether-ether-ketone (PEEK) and titanium-coated PEEK interbody fusion devices”.
3. Lebl, Darren R. MD, Cammisa, Frank P. Jr MD, Girardi, Federico P. MD, Wright, Timothy PhD, Abjornson, Celeste PhD. Spine 37(26):p 2151-2160, Dec. 15, 2012. “The Mechanical Performance of Cervical Total Disc Replacements In Vivo Prospective Retrieval Analysis of Prodisc-C Devices.”
4. Matthew Scott-Young, Evelyne Rathbone, Lauren Grierson; European Spine Journal (2022) 31:1273-1282. “Midterm osteolysis-induced aseptic failure of the M6-C™ cervical total disc replacement secondary to polyethylene wear debris.”
5. Manfred K. Muhlbauer, Ernst Tomasch, Wolfgang Sinz, Siegfried Trattnig, Hermann Stefan. Muhlbauer et al. BMC Musculoskeletal Disorders (2022) 23:227. “Cervical disc prostheses need a variable center of rotation for flexion extension below disc level, plus a separate COR for lateral bending above disc level to more closely replicate in-vivo motion: MRI-based biomechanical in-vivo study.”
6. Fatma Nur Depboylu, Evren Yasa; €Ozgu\ddot{ }r Poyraz; Joaquim Minguella-Canela; Feza Korkusuz; M Antonia De los Santos Lopez. Journal of Material Research and Technology, 2022; 17; 1408-1426. “Titanium based bone implants production using laser powder bed fusion technology.”

### Et al

7. Fayyazi, Amir H; Taormina Jennifer; Svach David: Stein Jeff and Ordway Nathaniel R. International Journal of Spine Surgery January 2015, 9 30; 14444/2030. “Assessment of Magnetic Resonance Imaging Artifact Following Cervical Total Disc Arthroplasty”
8. Veruva Sai Y.; Steinbeck Marla J.; Toth Jeffrey; Alexander Dominik D.; Kurtz Steven M. Clin Orthop Relat Res (2014) 472:3759-3769. “Which Design and Biomaterial Factors Affect Clinical Wear Performance of Total Disc Replacements?A Systematic Review.”
9. Kurtz Steven M.; Toth Jeffrey M.; Siskey Ryan; Ciccarelli Lauren; MacDonald Dan; Isaza Jorge; Lanman Todd; Punt Ilona; Steinbeck Marla; Goffin Jan; van Ooij Andre. Semin Spine Surg. 2012 Mar. 1; 24(1):57-70. “The Latest Lessons Learned from Retrieval Analyses of Ultra-High Molecular Weight Polyethylene, Metal-on-Metal, and Alternative Bearing Total Disc Replacements.”
10. McCombe Peter, Diwan Ashish and Wilke Hans-Joachim. Lumbar Spine Textbook»Section 3: Biomechanics»Section 3, Chapter 6: Biomechanics of Motion Preservation Technologies.
11. Wang Xiao-Fei, Meng Yang, Liu Hao, Wang, Bei-Yu and Hong Ying. J Orthop Surg Res2021 Jan 6; 16:12. “The impact of different artificial disc heights during total cervical disc replacement: an in vitro biomechanical study.”

I claim:

1. An artificial disc replacement implant comprising a cephalad endplate, a caudal endplate, and a core, wherein the core is disposed between the cephalad endplate and the caudal endplate,
wherein the cephalad endplate comprises a domed portion,
wherein the superior portion of the core has a convex surface configured to fit inside the domed portion of the cephalad endplate, and
wherein the superior surface of the cephalad endplate and the inferior surface of the caudal endplate comprise one or more textured or porous regions to facilitate bone ongrowth and/or ingrowth and one or more fixation elements.

2. The implant of claim 1 configured for insertion between two vertebrae in the cervical or lumbar region of the spine in a patient.

3. The implant of claim 2, wherein the caudal endplate has an anterior end and a posterior end, and wherein the anterior end has a height that is greater than the height of the posterior end, optionally wherein following insertion into the patient's spine a desired degree of lordosis is obtained.

4. The implant of claim 3, wherein caudal endplate has a lordotic angulation of about 0 degrees to about 9 degrees, from about 3 degrees to about 9 degrees, from about 3 degrees to about 6 degrees, or from about 6 degrees to about 9 degrees, optionally wherein the lordotic angulation is about 0 degrees, about 3 degrees, about 6 degrees, or about 9 degrees.

5. The implant of claim 2, wherein the one or more fixation elements are keels, spikes, or a combination thereof, wherein the one or more fixation elements comprise a sharp leading edge at the posterior aspect of the fixation element, and widen toward the anterior end of the implant, optionally wherein the leading edge at the posterior aspect of at least one of the fixation elements curves away from the posterior end toward the anterior end of the implant.

6. The implant of claim 5, wherein the fixation elements are two or more keels, optionally wherein the cephalad endplate comprises a first keel on a first side of the domed portion and a second keel on the opposite side of the domed portion, wherein the first and the second keels are symmetrically aligned relative to each other about a center line of symmetry (l) that runs anteriorly and posteriorly, and optionally a third keel, wherein the third keel is located adjacent to the posterior end of the cephalad endplate, and wherein the third keel is smaller that each of the first and second keels; and
optionally wherein the caudal endplate comprises a fourth keel and a fifth keel, wherein the fourth and fifth keels are symmetrically aligned relative to each other about a center line of symmetry (l) that runs anteriorly and posteriorly, and are located on opposite sides of the exterior surface and optionally a sixth keel, wherein the sixth keel is located adjacent to the posterior end of the cephalad endplate, and wherein the sixth keel is smaller that each of the fourth and fifth keels.

7. The implant of claim 6, wherein the first and second keels are aligned so that they diverge when viewed from the posterior end of the endplate to the anterior end of the endplate.

8. The implant of claim 7, wherein the angulation of each of the first and second keels relative to the center line (l) can range from 0.1° to 20°, 0.1° to 10°, 0.1° to 5°, 0.1° to 3°, 0.1° to 2.5°, 0.1° to 2°, 0.50 to 20°, 0.50 to 10°, 0.50 to 5° 0.50 to 3° 0.50 to 2.5°, 0.50 to 2°, 0.5° to 1°, 1° to 20°, 1° to 10°, 1° to 5°, 1° to 3°, 1° to 2.5°, or 1° to 2°.

9. The implant of claim 5, wherein the fixation elements are two or more spikes, optionally wherein the cephalad endplate comprises a first group of spikes on a first side of the domed portion and a second group of spikes on the opposite side of the domed portion, wherein the first and the second groups of spikes are substantially symmetrical, and wherein each group of spikes contains at least two spikes, optionally having different sizes, with the largest spike is located at the anterior end of the cephalad endplate; and
optionally wherein the caudal endplate comprises a third group of spikes and a fourth group of spikes, wherein the third and fourth groups of spikes are substantially symmetrical, and are located on opposite sides of the exterior surface, and wherein each group of spikes contains at least two spikes, optionally having different sizes, with the largest spike is located at the anterior end of the cephalad endplate.

10. The implant of claim 2, wherein the concavity of the inferior surface of the domed portion of the cephalad endplate matches the convexity of the superior portion of the core, such that the core fits inside the concavity in a ball-and-socket configuration.

11. The implant of claim 2, wherein the concavity of the inferior surface of the domed portion of the cephalad endplate is nonconcentric relative to the convexity of the superior portion of the core, such that the domed portion of the inferior surface of the cephalad endplate is elongated anteriorly and optionally also elongated posteriorly relative to the core, optionally each side of the domed portion of the inferior surface of the cephalad endplate is wider than the superior portion of the core when viewed in the mid coronal plane.

12. The implant of claim 11, wherein when the implant is in neutral alignment and is viewed from the mid sagittal view, the anterior elongation of the inferior surface of the domed portion relative to the superior portion of the core has a first gap (g₁),
optionally the posterior elongation of the inferior surface of the domed portion relative to the superior portion of the core has a second gap (g₂), and
optionally when the implant in neutral alignment and is viewed in the mid coronal plane, the domed portion is offset laterally on both sides from the superior portion of the core, such that a third and fourth gap, g₃ and g₄, exist on each side the inferior surface of the domed portion relative to the of the superior portion of the core.

13. The implant of claim 12, wherein the implant is a cervical disc replacement implant, and wherein the first gap (g₁) has a length of at least 0.1 mm, optionally in the range of 0.1 to 2 mm, 0.1 to 1 mm, 0.3 to 2 mm, 0.3 to 1 mm, 0.5 to 2 mm, 0.5 to 1 mm, 0.5 to 0.9 mm, 0.5 to 0.8 mm, or 0.5 to 0.7 mm, optionally 0.6 mm, 0.61 mm, 0.62 mm, 0.63 mm, 0.64 mm, 0.65 mm, 0.66 mm, 0.67 mm, 0.68 mm, 0.69 mm, or 0.7 mm.

14. The implant of claim 13, wherein the second gap (g₂) is present and has a length of at least 0.1 mm, optionally in the range of about 0.1 to 0.5 mm, 0.1 to 1 mm, 0.1 to 0.4 mm, 0.1 to 0.3 mm, or 0.1 to 0.35 mm, optionally 0.3 mm, 0.31 mm, 0.32 mm, 0.33 mm, 0.34 mm, or 0.35 mm.

15. The implant of claim 14, wherein the third and fourth gaps (g₃ and g₄) are present and independently each of the third and fourth gaps has a length of at least 0.1 mm, optionally in the range of about 0.1 to 1 mm, 0.1 to 0.5 mm, 0.1 to 0.4 mm, 0.1 to 0.3 mm, or 0.1 to 0.25 mm, optionally wherein the lengths of the third and fourth gaps are the same or substantially the same.

16. The implant of claim 12, wherein the implant is a lumbar disc replacement implant, and wherein the first gap (g₁) has a length of at least 0.2 mm, optionally in the range of about 0.2 to 3 mm, 0.2 to 2 mm, 0.6 to 2 mm, 0.6 to 3 mm, 1 to 3 mm, 1 to 2 mm, 1 to 1.8 mm, 1 to 1.6 mm, or 1 to 1.4 mm, optionally 1.1 mm, 1.2 mm, 1.22 mm, 1.24 mm, 1.26 mm, 1.28 mm, 1.3 mm, 1.32 mm, 1.34 mm, 1.36 mm, 1.38 mm, or 1.4 mm.

17. The implant claim 16, wherein the second gap (g₂) is present and has a length of at least 0.1 mm, optionally in the range of about 0.1 to 2 mm, 0.1 to 1 mm, 0.1 to 0.9 mm, 0.1 to 0.8 mm, 0.1 to 0.7 mm, 0.4 to 0.7 mm, or 0.6 to 0.7 mm, optionally 0.6 mm, 0.61 mm, 0.62 mm, 0.63 mm, 0.64 mm, 0.65 mm, 0.66 mm, 0.67 mm, 0.68 mm, 0.69, or 0.7 mm.

18. The implant of claim 17, wherein the third and fourth gaps (g₃ and g₄) are present and independently each of the third and fourth gaps has a length of at least 0.2 mm, optionally in the range of about 0.2 to 2 mm, 0.2 to 1 mm, 0.2 to 0.8 mm, 0.2 to 0.6 mm, or 0.2 to 0.5 mm, optionally wherein the lengths of the third and fourth gaps are the same or substantially the same.

19. A method for replacing a damaged cervical or lumbar disc in a patient comprising:
after the damaged disc is removed from between a first and second vertebral body, inserting the implant of any one of claim 2 into the space between the first and second vertebral bodies.

20. The method of claim 19, wherein the method does not include a chiseling and/or drilling step to prepare the surfaces of the vertebral bodies prior to inserting the implant therebetween.
